(19) United States
(12) Patent Application Publication
(10) Pub. No.: US 2016/0073961 A1
(43) Pub. Date: Mar. 17, 2016

(54) METHOD AND SYSTEM FOR CHARACTERIZING SPORTING ACTIVITIY

(71) Applicants: Cheng Lock Donny SOH, Singapore (SG); Chee How LEE, Singapore (SG)

(72) Inventors: Donny Cheng Lock SOH, Singapore (SG); Chee How LEE, Singapore (SG)

(21) Appl. No.: 14/888,050

(22) PCT Filed: Apr. 28, 2014
(86) PCT No.: PCT/SG2014/000187

§ 371 Date: Oct. 29, 2015

Related U.S. Application Data
(60) Provisional application No. 61/817,547, filed on Apr. 30, 2013.

(51) Int. Cl. A61B 5/00; A61B 5/11

(52) U.S. Cl. CPC A61B 5/6895; A61B 5/1123; A61B 5/7246; A61B 5/743; A61B 5/0022; A61B 2503/10; A61B 2560/0475

(57) ABSTRACT

A system is proposed for characterizing the performance of a sportsman. The system is capable of using motion data it receives describing a movement during the sport, to distinguish the type of sport, and type of stroke, which is being played. The system can furthermore detect the quality of a stroke involving an impact (e.g. an impact between an item of sports equipment and a ball), based on the observation that a good stroke will be in a “sweet spot” in which the amount of vibration caused by the impact is limited.

### FIELD OF THE INVENTION

The invention relates to methods and systems for characterizing sporting activity. The sport may be one involving an item of sports equipment, such as a tennis racket or a golf club.

### BACKGROUND OF THE INVENTION

It is known to detect the motion of an item of sports equipment to characterize the usage of the sporting equipment. In some systems this is done by providing a motion sensor on the item of sporting equipment. For example, U.S. Pat. No. 7,870,790 describes a method of evaluating the feeling of hitting a golf club. The vibration of the golf club in the circumferential direction of the shaft of the golf club when a golf ball is struck, is measured by a three-axis accelerometer. The vibration is analyzed by a hand-arm vibration measurement filter, to evaluate the feeling of hitting the golf club. The purpose of this is to provide an objective method of comparing the quality of different forms of golf club. In other systems the motion of the sporting equipment is measured by external motion detection devices. In U.S. Pat. No. 7,938,733, for example, the motion of a golf club is detected by a light sensor located on the stomach of a golfer, which measures the motion of an illuminator device located on the club.

### SUMMARY OF THE INVENTION

A first aspect of the invention proposes a system and method for characterizing a movement (i.e. a motion) during performance of a sport. Typically, the movement is that of an item of sports equipment, but it may alternatively be a movement of a body part of the player of the sport. The system is capable of distinguishing, from motion data it receives describing the movement, the type of sport which produced the movement, and/or which stroke type for a given sport produced the movement.

For example, if the system receives motion data describing the movement of an item of sports equipment, it may be able to determine whether the movement is part of one of a predetermined set of sports, the set preferably including at least one of tennis and golf.

In a further example, if the system receives motion data describing the movement of an item of sports equipment, it may be able to determine if the movement was a forehand or a backhand stroke by a tennis racket, or a drive or a putt by a gold club.

To put this more generally, the system is able to determine which of a predetermined set of strokes was being carried out when the motion data was generated, the predetermined set of strokes including strokes from different sports and/or different strokes from a single stroke. Preferably, the predetermined set of strokes includes, for each of a plurality of different sports, a plurality of strokes for each sport.

In a second aspect of the invention, the system detects the quality of a stroke made by an item of sports equipment, and involving an impact (e.g. an impact between the item of sports equipment and a ball). The stroke may be one of which the type has been identified by the first aspect of the invention. The system distinguishes a good stroke from a bad one by deriving a vibration parameter indicating the number of vibrations in the stroke (i.e. the number of times that an acceleration value passes through zero).

This aspect of the invention is based on the observation that a good stroke will be in a “sweet spot” in which the amount of vibration caused by the impact is limited. One way of expressing this is that the number of vibrations which occurs within a given time window does not exceed a threshold. The threshold will be different for sports and for different strokes. By contrast, a bad stroke—i.e. one which is off centre—will typically cause a jarring motion in which the number of vibrations is greater.

In a preferred form of the invention, the system involves three items. First, there is a motion sensor device for attachment to the item of sporting equipment or to the player (e.g. to his or her clothing). Second, there is a mobile device (such as a phone, laptop or dedicated receiving device) which is in wireless communication with the motion sensor device and receives the motion data. Third, there is preferably a remote computing apparatus (a backend computational platform) in communication with the mobile device. The analysis of the motion data is performed by one of the mobile device or the remote computing device, or may be split between them. The mobile device may carry application software for this purpose. In one form, the mobile device provides enough analysis to give instant feedback to the player of the sports equipment, but communicates data to a remote computing device such that the remote computing device can do further analysis (e.g. to generate statistics describing multiple strokes), or make it available for sharing to other individuals. For example, this makes possible a personal logbook, and leaderboard comparing different individuals.

Furthermore the system may be able to generate numerical information, such as calories burnt or energy usage.

### BRIEF DESCRIPTION OF THE FIGURES

By way of example only, an embodiment of the invention will be described with reference to the accompanying drawings, in which:

FIG. 1, which is composed of FIGS. 1(a) to 1(c), is a schematic drawing of a motion sensor device for use in an embodiment of the present invention;

FIG. 2 is a flow diagram of the operation of the system.

FIG. 3, which is composed of FIGS. 3(a) and 3(b), shows data demonstrating the difference between motion data describing a sweet spot stroke and a bad stroke; and

FIG. 4, which is composed of FIGS. 4(a) and 4(b), shows vibration parameters calculated respective from five sweet spot strokes and five bad strokes.

### DETAILED DESCRIPTION OF THE PREFERRED EMBODIMENT

One embodiment of the invention is a system which includes a motion sensor device and a mobile device which communicates, preferably wirelessly, with the motion sensor device.

The motion sensor may be releasably attached to an item of sports equipment, such as a golf club or tennis racket, such as by straps, bands, clasps or clips.

Alternatively, the motion sensor may be worn by the athlete, e.g. be attachment to the athlete by a band, or by attachment to part of the athlete's clothing, e.g. his or her shoe.

The motion sensor may have one or more of the following sensors for generating motion data: accelerometers, magnetometers, gyroscopes and/or a GPS unit.

Typically, the motion sensor further comprises a transmitter for communicating detected data. Preferably, the motion sensor comprises a transmitter that uses Bluetooth low energy (BLE) standard. BLE is chosen over other conventional wireless communication protocols such as RF, Bluetooth, NFC) because of its low energy and pervasiveness.

According to a particular example, the integrated circuits (chips) used in the motion sensor are CC2540/CC2541 (Texas instruments) and LSM330DLC chip (ST Microelectronics).

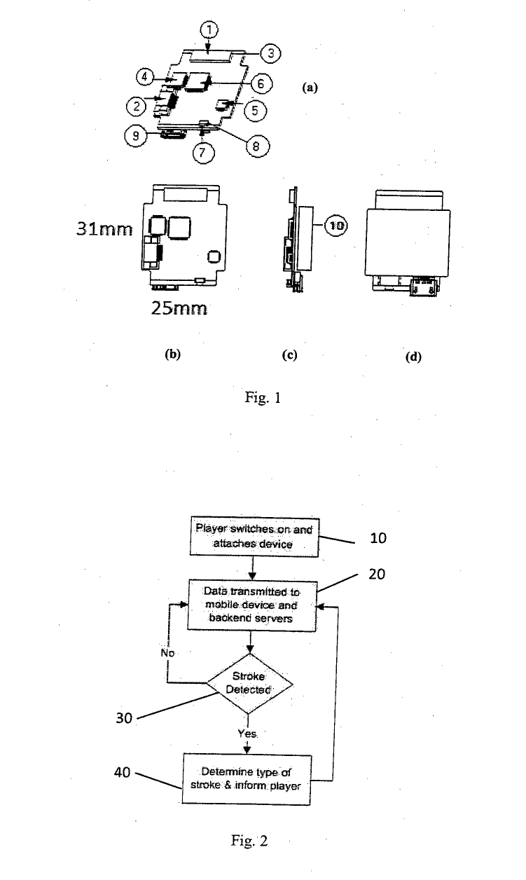

FIG. 1 shows a motion sensor device which may be used in the embodiment. The motion sensor is pictured in perspective view (FIG. 1 (a)), top plan view (FIG. 1(b)), side view (FIG. 1(c)) and bottom plan view (FIG. 1(d)). The motion sensor device has (as shown in PCB 3) an antenna 1, a battery 10, a battery charger 5, an accelerometer and/or a gyroscope 4, a switch 8 to turn the device on and off and to turn the BLE transmitter 6 on and off, a reset button which is used if the sensor becomes unresponsive and the switch it unable to switch it on or off, connectors 2, 9, and an LED 7. The LED 7 lights up red when the motion sensor device is on and searching for another Bluetooth-enabled device to connect to, and turns green when one is found.

The mobile device may be a mobile phone, tablet, or any display devices (e.g. Google glasses). It receives a series of motion readings from the motion sensor device, and analyses them by the following methods.

Optionally, some of the calculation may be done by a remote computing device (server) in communication with the mobile device. The remote server may act as a platform where data generated by the motion sensor (or further data derived from it, such as data describing the results of analyzing the data generated by the motion sensor) is stored. This data may be accessed by other computing devices, for example by an application programming interface (API) running on the server. This allows programmers to write applications running on the other computing devices which connect to the server via the API. Such programmers may optionally subscribe to a service provided by the owner of the server. The service allows them to connect to the server, to download the data from the motion sensor and/or data obtained from it by analysis, and optionally to perform additional analysis of it.

The flow diagram of the system is shown in FIG. 2. In step 10, the player (athlete) turns on the system, and attaches it to an item of sports equipment wielded as part of the performance of the sport (e.g. a golf club, tennis racket, or golf glove), or to his or her body or clothing.

In step 20, the motion sensor device continually generates motion readings, and sends them to the mobile device which optionally transfers them to the backend server. In step 30, mobile device and/or backend server analyses them to detect a stroke. For example, if the motion readings are accelerations, it can be determine whether the sum of the motion readings during a certain time includes an acceleration above a threshold (if the motion readings are not accelerations, it can determine if they imply an acceleration above the threshold). In step 40, it uses the methods described below to determine the type of stroke and analyze its quality. The mobile device then generates a message to the player, giving him feedback on the stroke. The system, then loops back to step 20, until the system is turned off.

We now turn to an explanation of the methods carried out in step 40.

(i) Stroke Classification/Detection

This is performed by producing an information digest from the received series of motion readings. This information digest, is compared with a plurality of pre-generated templates to select one which most closely matches the information digest. Each template is associated with a corresponding type of stroke in a corresponding sport. The motion is recognized as being the type of stroke corresponding to selected template.

(a) Pre-Generation of the Templates

Each template was pre-generated using a plurality of examples of strokes of the corresponding stroke. Suppose, for example that we are creating a template for “club face in” strokes of a golf club. A large number (e.g. 100) of example “club face in” strokes are performed. For each stroke a corresponding set of motion readings is obtained, and the sets of motion readings are labeled as being “club face in” strokes. Note that the sets of motion readings may be collected starting at time when a stroke has been automatically detected, or at a later time (e.g. 2 seconds later). A template for “club face” in strokes is generated from the sets of motion readings.

First, each set of motion readings is converted into an information digest referred to as a Symbolic Aggregate approXimation form, or “SAX form” [1]. In a first step, each set of motion readings is divided into a plurality of time windows. For each of the plurality of windows, there is a dimensionality reduction or compression step. For example, all motion readings within the time window are compressed into a single value (“summary value”) such as the mean, variance, or another basic statistical parameter.

The system uses predefined ranges for the statistical parameter, such that the value of the statistical parameter is equally likely to fall into any of the ranges. For example, is during a hit the acceleration ranges from +100 to +5, we will divide this range up into equally probable sections based on the distribution. Suppose for example, that there were 10 systems of stroke to be recognized, and for each type there were 20 example strokes, and for each of these there were 30 time windows, the distribution would be found of all the values taken by the parameter in these 10×20×30 windows, and the ranges would be found based on this distribution. Each of these ranges is associated with a respective symbol from a “vocabulary” (i.e. set of symbols). For each example stroke, the single value obtained for each time window is converted into the respective symbol, according to the range into to which the corresponding single values falls. In other words, each example stroke is re-written as a string of symbols, with the number of symbols equal to the number of time windows. This constitutes the information digest (SAX form).

A template for “club face in” strokes is then generated from the SAX forms for the example “club face in” strokes. One way of doing this is to generate a large number of candidate templates randomly, in the form of strings of the symbols. We then find which of the candidate templates has the lowest average Euclidean distance to the SAX forms for the example “club face in” strokes, and select this candidate template as the template for “club face in” strokes. The “Euclidean distance” between two strings of symbols (e.g. a string presenting a SAX form and an string representing a template) may be defined as the sum over the components of the first string of the modulus of the difference between the component of the first string and the corresponding component of the second string (for example, if the first string is 1, 5, 7, 5 and the second string is 1, 6, 7, 6, then the Euclidean distance is |4−4|+|5−6|+|7−7|+|4−5|=2, that is the pairwise modulo difference between the sets of symbols.

Similarly, a large number (e.g. 100) of example “club face out” strokes are generated, and for each such example stroke a corresponding set of motion readings is obtained and labeled as a “club face out” stroke. A template for “club face out” strokes is generated from the SAX forms of the sets of motion readings for the corresponding example strokes.

This process is continued to produce respective templates for many different sorts of strokes, in many different sports.

Generally, different types of stroke involve different movements. Thus, each type of stroke exhibits unique/fingerprinting characteristics in terms of its waveform. This is captured by the templates. For example, a template representing forehand tennis strokes will be very different from that representing backhand tennis strokes. The templates may be referred to as “SAX templates”.

The generation of the templates may be considered as a form of “supervised learning”, since it is performed based on a number of examples of each type of stroke, and each template is generated based only on the examples of the corresponding type of stroke.

Note that the pre-generation of the templates may be done by the manufacturer of the embodiment (i.e. it is not player specific). Alternatively, the process described above may be done by the player during a set-up process for the embodiment, using examples of each stroke performed by the player. This may make the embodiment more accurate, because it is not undermined by the fact that different individuals may perform a given stroke in different ways. It also has the advantage that the embodiment can be used to recognize a new sort of stroke (e.g. a stroke in a sport not envisaged by the manufacturer).

(b) Using the Pre-Generated Templates in the Embodiment

The set of motion data captured by the motion sensor device during a period after a stroke has been detected (e.g. by detecting an acceleration being above the threshold) is converted into the SAX form, as described above. As mentioned above, that period may begin a predetermined time after the stroke is detected, e.g. 2 seconds later. That SAX form is compared with each of the pre-defined templates of different types of known stroke, and most similar template is selected. To determine the extent of similarity between the data and a plurality of templates, a Euclidean distance (referred to as a “SAX feature”) is computed between each template and the SAX form obtained based on the motion data. The most closely matching template (i.e. template with the lowest SAX feature) is selected. This is defined as the template having the smallest Euclidean distance from the SAX form obtained based on the motion data. The motion data is recognized as having being created from a stroke of the type associated with the most closely matching template (e.g. if the most closely matching template is the “club face out” template, then the stroke is recognized as a golf stroke, and in particular as a “club face out” stroke). In other words, by analyzing the data generated by the motion sensor device for a particular movement, the corresponding type of sports may be identified and/or the corresponding type of strokes of that sport.

Various statistics of the movements may be generated to monitor the athlete's performance. For example, such statistics may include the number of forehand strokes, backhand strokes and etc.

Conveniently, the motion detected by the motion sensor may be the motion of an items of sports equipment (such as a golf club, or tennis racket), but the method may also be used for non-racquet sports, especially for those sports which involve repetitive movements or requiring the analysis of strength and sudden impulses, such as fencing or boxing.

(ii) Sweet Spot Detection

We now describe a further method which can be performed in step 40. This method is applicable in the case that the stroke is determined to be in a sport involving an impact, such as an impact with a ball. It is particularly relevant when the sport is one in which an item of sporting equipment (e.g. a golf club or racquet) impacts an object (e.g. a ball) the method can characterize the movement of the item of sports equipment leading to the impact.

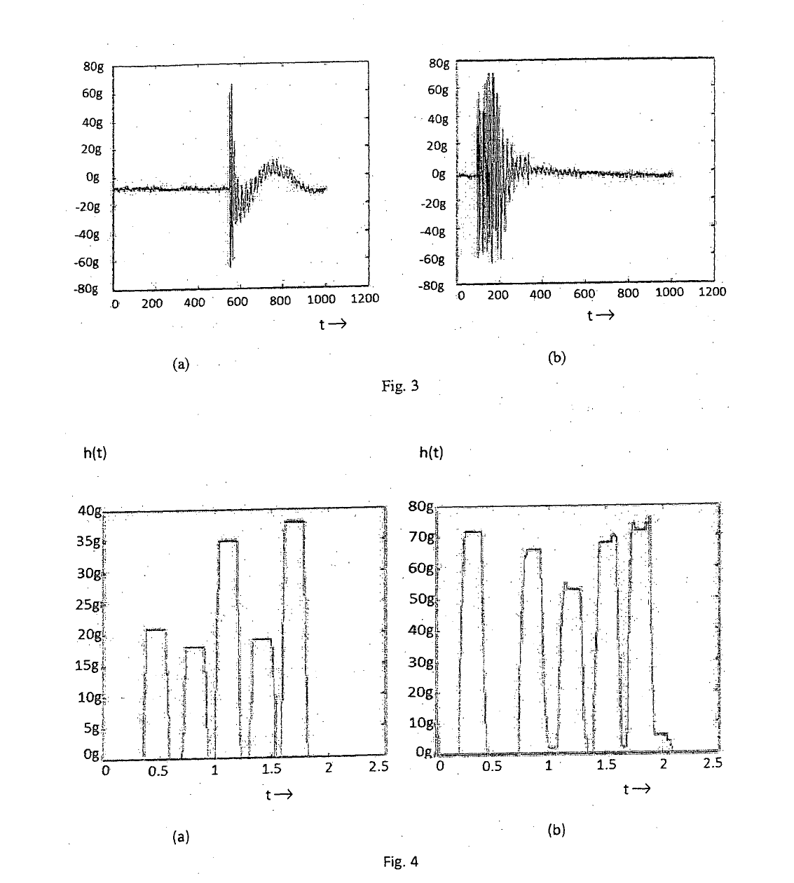

In a particular example, the method detects if a particular stroke hit the object on a “sweet spot”. Specifically, data describing the movement may be analyzed and characterized as either an on-center or off-center stroke. This is done by counting the number of vibrations within a certain window, and comparing it to a threshold. Typically, the threshold is specific to the type of stroke. The reason for counting the number of vibrations can be seen by comparing the pattern of motion data expected for an on-centre shot (as shown in FIG. 3(a)) with the pattern of data expected for an off-centre shot (as shown in FIG. 3(b)). FIG. 3(a) shows smooth vibration patterns for a hit on the sweet spot, whereas FIG. 3(b) shows a jarring vibration pattern for a non-sweet spot hit. The vertical axis shows acceleration in units of “g”, the gravitational constant.

The method uses a series of motion readings f(t) from the motion sensor device. Each motion reading indicates the acceleration (e.g. experienced by an item of sports equipment) at a sampling point t during or after the impact of the item of sports. For a given window starting at a time t and including a number of time samples equal to W (i.e. time samples t to t+W) we calculate a parameter h(t) using the formula:

$$h ( t ) = {\sum }_{{t}^{′} = t}^{{t}^{′ = t + W}} g ( {t}^{′} ) where \begin{matrix}g ( t ) = 1 & f ( t ) * f ( t + 1 ) < 0 \\ g ( t ) = 0 & otherwise\end{matrix} \qquad ( 1 )$$

g(t) has the value of 1 if, and only if, there is a change in the direction of acceleration from time t to time t+1. h(t) is a vibration parameter, indicating the number of times at which the acceleration becomes zero (“zero crossings”) during a window of W time units starting at time t.

Note that the spacing of the time samples should be much less than the period of the resonant frequency of the item of sports equipment, otherwise the sampling may be subject to undesirable resonance effects.

If the score h(t) is above a threshold value of X for any time t during the stroke, the stroke is judged to be off centre. Otherwise, it is considered as on-centre. This result may be fed-back to the player by the mobile device and/or statistics concerning the number of on-centre/off-centre hits may be accumulated, e.g. at the remote computing device.

The values of W and X may be determined empirically during the training process. During the training process, strokes will be hit deliberately on/off centre to simulate good/bad strokes. This is a supervised learning process.

For example, FIG. 4 shows an experiment conducted for the sport of tennis. The sliding window is 2000 time units. FIG. 4(a) shows an example in which five sweet spot hits are made on the tennis racquet. FIG. 4(b) shows an example in which the five hits are not on the sweet spot. Each of FIGS. 4(a) and 4(b) includes five distinct peaks, corresponding to the five strokes. Note than the highest peak in FIG. 4(a) is lower than the lowest peak in FIG. 4(b). This shows that h(t) is a useful parameter for distinguishing sweet spot hits from other hits. The threshold may be chosen as a value in between these two peak values.

Note that in FIG. 3(a) quite a large portion of the waveform is spaced away from the zero acceleration position. This means that the vibrations in those parts of the waveform do not contribute to the value of h(t) given by Eqn. (1). This is not a problem in the example above, since it makes the value of h(t) even lower, but it is possible to imagine situations (e.g. certain types of sport) in which the waveform from a non-sweet spot hit would similarly include a significant portion which is spaced apart from the zero acceleration position. If so, even though the amplitude of the vibrations may be undesirably large, they may not cause the acceleration value to move through zero, and therefore do not increase the value of h(t) as defined by Eqn. (1). In other words, the value of h(t) might not, in this give, give a value which is above the threshold, and therefore be indicative of the non-sweet spot hit. One way of avoiding this problem would be to form a moving average f′(t) of the waveform f(t) (e.g. at each time t the average could be found of the value of f(t) at each of a corresponding predetermined range of t values, e.g. the previous 5 values of t), and then to form h(t) by counting the number of times that f(t) crosses f′(t). For example, h(t) could be calculated as:

$$h ( t ) = {\sum }_{{t}^{′} = t}^{{t}^{′ = t + W}} g ( {t}^{′} ) where \begin{matrix}g ( t ) = 1 & ( f ( t ) - {f}^{′} ( t ) ) * ( f ( t + 1 ) - {f}^{′} ( t ) ) < 0 \\ g ( t ) = 0 & otherwise\end{matrix} \qquad ( 2 )$$

(iii) Derivation of Numerical Parameters Describing a Stroke

Step 40 of the flow of FIG. 2 may also include deriving numerical parameter describing a stroke. This data may be provided to the player to assess their performance. Non-exhaustive examples of such information are described below.

### 1. Force Generated by the Stroke

Biomechanical studies of the musculoskeletal system show that a single arm is approximately 5.33% of the entire body mass [2]. For example, if the body mass of a person is 70 kg, the mass of his arm may be approximated as 3.73 kg. Let mₐᵣₘ denote the mass of an arm, m_{rac} denote the mass of a racquet (tennis, golf club, etc), and a denote the acceleration, of the racquet measured by the motion sensor, the force generated may be calculated as

F=(m_{rac}+mₐᵣₘ)*a

Assuming the maximum acceleration (aₘₐₓ) occurs just before the impact, aₘₐₓ may be obtained during the time window of t_{w} units before the impact, as discussed earlier. Hence, the maximum amount of force generated may be calculated by:

F=(m_{rac}+mₐᵣₘ)*aₘₐₓ.

2. Velocity of Object after Impact

The velocity of the racquet may be estimated using:

$${v}_{racq} = {u}_{0} * t + \frac{1}{2} * {a}_{max} * {t}^{2}$$

Assuming u₀ is zero and t is the length of the time window t_{w}, then

$${v}_{racq} = \frac{1}{2} * {a}_{max} * {t}_{w}^{2}$$

3. Kinetic Energy of the Object after Impact

The basic formula for finding of kinetic energy is:

$$e = \frac{1}{2} * m * {v}^{2}$$

Hence in this case, the energy is

$${e}_{racq} = \frac{1}{2} * ( {m}_{rac} + {m}_{arm} ) * {v}_{racq}^{2}$$

Assuming a fraction, given by the coefficient of restitution (COR—a measure of the energy loss or retention when two objects collide), of the energy of the racquet is transferred to the ball, the energy of the projectile object may be calculated by:

eₚᵣₒⱼ=e_{racq}*COR

Hence, to calculate the velocity by this formula, we use

$${ε}_{proj} = \frac{1}{2} * {m}_{proj} * {v}_{proj}^{2} .$$

Whilst example embodiments of the invention have been described in detail, many variations are possible within the scope of the invention as will be clear to a skilled reader.

### REFERENCES

[1] Pekka Siirtola, Heli Koskimäki, Ville Huikari, Perttu Laurinen, and Juha Röning. Improving the classification accuracy of streaming data using SAX similarity features. Pattern Recogn. Lett. 32, 13 (October 2011)
[2] Plagenhoef, S., Evans, F. G. and Abdelnour, T. Anatomical data for analyzing human motion. Research Quarterly for Exercise and Sport 54, 169-178. 1983

1. A method of characterizing a movement during the performance of a sport, using a motion sensor releasably attached to an item of sports equipment to generate motion data describing the movement, the method comprising the steps of:
(i) receiving a series of motion readings from the motion sensor, wherein each motion reading describes the movement at a sampling point during the movement;
(ii) producing an information digest from the received series of motion readings;
(iii) comparing the information digest against each of a plurality of pre-determined templates, to select a most closely matching template from the plurality of templates, wherein each of said plurality of templates describes a respective type of stroke, wherein the pre-determined templates include templates which describe a respective plurality of strokes in the same sport; and
(iv) characterising the movement as the stroke corresponding to the most closely matching template.

2. A method according to claim 1 in which the pre-determined templates include templates which describe respective strokes of different sports.

3. The method of claim 1 wherein step (ii) comprises the step of dividing the received series of motion readings into a plurality of windows, and, for each of the plurality of windows, applying a dimensionality reduction technique on the motion readings of the window to produce a summary value.

4. The method of claim 3 wherein step (ii) further comprises the step of assigning a symbol of a vocabulary to each summary value, and forming an information digest comprising a sequence of said symbols corresponding to said summary values across said plurality of windows, wherein each said symbol corresponds to respective range of the summary value, said each range being chosen such that the probability of the appearance of each symbol within the vocabulary is equal.

5. The method of claim 1, wherein step (iii) comprises the step of computing for each template of the plurality of templates a Euclidean distance with the information digest, and selecting the most closely matching template as the template having the smallest Euclidean distance from the information digest.

6. The method of claim 1, wherein each template is obtained from sample data describing a plurality of examples of the corresponding type of stroke.

7. A method of characterising a movement during the performance of a sport, the movement including an impact, the method using a motion sensor releasably attached to an item of sports equipment to generate motion data describing an acceleration which is part of the movement, the method comprising the steps of:
(i) receiving a series of motion readings from the motion sensor, each motion reading being indicative of the acceleration during or after the impact;
(ii) computing a score using a window defined over a part of the series of motion readings, the score being indicative of a number of zero-crossings in the motion readings within the window; and
(iii) characterising a quality of the impact by comparing the score against a threshold value comprising characterising the impact as an off-center stroke if the score is greater than the threshold value.

8. The method of claim 7 wherein step (ii) comprises detecting the presence of a zero-crossing based on the values of two consecutive motion readings within the window.

9. The method of claim 8 wherein step (ii) further comprises computing a sum of the zero-crossings within the window based on the values of every two consecutive motion readings within the window, said sum being the score.

10. The method of claim 7 wherein the method further comprises the steps of:
(iv) displacing the window to cover a further part of the series of motion readings; and
(v) repeating steps (ii) and (iii) using the displaced window.

11. The method of claim 7 wherein the method further comprises the steps of:
(iv) deriving a maximum acceleration value from the series of motion readings; and
(v) calculating a velocity of an item of sports equipment using the maximum acceleration value.

12. The method of claim 11 wherein the method further comprises the steps of:
(vi) calculating an energy of the item of sports equipment based on the velocity of the item of sports equipment;
(vi) calculating an energy of the object after the impact based on the calculated energy of the item of sports equipment; and/or
(vii) calculating a velocity of the object after the impact based on the calculated energy of the object with a mass of the object.

13. A computer for analyzing a movement of an item of sports equipment using a motion sensor device releasably attached to the item of sports equipment, the computer comprising:
a receiver configured to receive a series of motion readings from the motion sensor device, and
a processor containing software configured to carry out—a method of characterizing a movement during the performance of a sport, using a motion sensor releasably attached to an item of sports equipment to generate motion data describing the movement, the method comprising the steps of:
(i) receiving a series of motion readings from the motion sensor, wherein each motion reading describes the movement at a sampling point during the movement;
(ii) producing an information digest from the received series of motion readings;
(iii) comparing the information digest against each of a plurality of pre-determined templates, to select a most closely matching template from the plurality of templates, wherein each of said plurality of templates describes a respective type of stroke, wherein the pre-determined templates include templates which describe a respective plurality of strokes in the same sport; and
(iv) characterising the movement as the stroke corresponding to the most closely matching template.

14. A server system comprising a server in communication with a relay device, the relay device being for communication with a motion sensor device releasably attached to an item of sports equipment to receive a series of motion reading from the motion sensor device,
at least one of the server and relay device containing software configured to carry out a method of characterizing a movement during the performance of a sport, using a motion sensor releasably attached to an item of sports equipment to generate motion data describing the movement, the method comprising the steps of:
(i) receiving a series of motion readings from the motion sensor, wherein each motion reading describes the movement at a sampling point during the movement;
(ii) producing an information digest from the received series of motion readings;
(iii) comparing the information digest against each of a plurality of pre-determined templates, to select a most closely matching template from the plurality of templates, wherein each of said plurality of templates describes a respective type of stroke, wherein the pre-determined templates include templates which describe a respective plurality of strokes in the same sport; and
(iv) characterising the movement as the stroke corresponding to the most closely matching template;
the server storing data obtained using the series of motion readings from the motion sensor device, and presenting an interface for accessing the stored data, whereby further computing devices may obtain access to the stored data using the interface.
